(19) United States
(12) Patent Application Publication
(10) Pub. No.: US 2021/0378857 A1
(43) Pub. Date: Dec. 9, 2021

(54) FLOW STOPPER PREVENTING BACKFLOW OF URINE

(71) Applicant: SINGSILVER HEALTH PTE. LTD., Dongguan, Guangdong (CN)

(72) Inventors: Chung Hing CHENG, Dongguan (CN); Kam Tim YEUNG, Dongguan (CN)

(21) Appl. No.: 17/287,996

(22) PCT Filed: Oct. 26, 2018
(86) PCT No.: PCT/CN2018/112033

§ 371 Date: Apr. 23, 2021

(51) Int. Cl. A61F 5/44; A61M 39/24; A61M 39/12; A61M 25/00

(52) U.S. Cl. CPC A61F 5/4405; A61M 39/24; A61M 39/12; A61M 2039/1038; A61M 2205/0205; A61M 2039/2433; A61M 25/0017

(57) ABSTRACT

A flow stopper preventing the backflow of urine comprises a urinary catheter connector, a flow stopping valve, and a urinary bag connector. The flow stopping valve is a membrane material. A flow channel runs through the front and rear ends of the flow stopping valve. The flow channel is provided with a front-end inlet and a rear-end outlet. The front-end inlet is in communication with a valve port of the urinary catheter connector. The rear-end outlet is a flat slit. A channel of the urinary bag connector is provided with a front-end port and a rear-end port connected to a tube of a urinary bag. The flow stopping valve is arranged within the channel. Moreover, the rear-end outlet of the flow channel faces the rear-end port of the channel. The flow stopper can prevent the backflow of the urine and prevent a patient from being infected.

### BACKGROUND OF THE INVENTION

### 1. Field of the Invention

The present invention relates to a medical device, and more particularly to a flow stopper preventing backflow of urine.

### 2. Description of the Prior Art

A urine collector is a medical device commonly used in clinic, mainly including a urinary catheter, a urine bag, and a flow stopper. When this urine collector is used to collect urine from patients who cannot urinate normally, the urinary system may be infected. According to relevant reports, urinary system infections account for about 60%-70% of iatrogenic infections. 2%-4% of infections caused by urinary catheters develop gram-negative bacteria sepsis. Every year about 30,000 people worldwide die from urinary system infections caused urinary catheters. Therefore, a conventional urine collector is provided with a flow stopper to prevent urine from flowing back.

The patient's urine is drained out of the bladder through a urinary catheter and a choke to be temporarily stored in a urine bag. However, if the bag is elevated above the patient's urethra, gravity causes the urine to flow back into the bladder. Therefore, the patient may be particularly susceptible to repeated urinary infections. The choke of the conventional flow stopper is closed manually to reduce the chance of the backflow of urine. The flow stopper of the prevent invention can be automatically closed to prevent the backflow of urine, and is applied with an antibacterial coating to further reduce the chance of urinary infections.

### SUMMARY OF THE INVENTION

In view of the defects of the prior art, the primary object of the present invention is to provide a flow stopper to prevent backflow of urine. It effectively solves the problem that the conventional flow stopper is closed manually and can prevent urine from flowing back.

In order to achieve the above object, the present invention adopts the following technical solutions:

A flow stopper comprises a urinary catheter connector, a flow stopping valve, and a urinary bag connector.

The urinary catheter connector includes a base, a urinary catheter port extending from an outer side of the base, and a valve port extending from an inner side of the base. The urinary catheter port is in communication with the valve port.

The flow stopping valve is made of a membrane material. Inner and outer surfaces of the flow stopping valve are coated with a bactericidal substance. The flow stopping valve has a flow channel passing through front and rear ends of the flow stopping valve. The flow channel is provided with a front-end inlet and a rear-end outlet. The front-end inlet is in communication with the valve port. The rear-end outlet is a flat slit.

The urinary bag connector has a channel passing through front and rear ends of the urinary bag connector. The channel is provided with a front-end port and a rear-end port connected to a tube of a urinary bag. The base is hermetically connected to the front-end port. The flow stopping valve is located in the channel. The rear-end outlet of the flow channel faces the rear-end port of the channel.

Preferably, the flow stopping valve is an integrally formed structure.

Preferably, the flow stopping valve includes a first flow stopping valve and a second flow stopping valve that are in communication with each other. The flow channel is formed inside the first flow stopping valve and the second flow stopping valve. A front-end inlet of the first flow stopping valve is in communication with the valve port. A rear-end outlet of the first flow stopping valve is a flat slit. The second flow stopping valve has a flat shape. The second flow stopping valve is sleeved onto the rear-end outlet of the first flow stopping valve.

Preferably, a junction of an interior of a front end and an interior of a rear end of the first flow stopping valve is Y-shaped.

Preferably, the urinary catheter connector is a pagoda-shaped connector. An outer surface of the base has an external thread. An inner wall of the front-end port has an internal thread. The external thread is mated with the internal thread.

Preferably, the valve port has a cylindrical shape. The front-end inlet of the flow channel has a circular shape. The front-end inlet is sleeved onto the valve port.

Preferably, the urine catheter connector and the urine bag connector are made of a bactericidal material.

Preferably, the urine bag connector includes a body portion and a tail portion integrally extending from a rear end of the body portion. The front-end port is located on the body portion. The rear-end port is located on the tail portion. An inner wall of the tail portion has an internal thread connected with the tube of the urine bag.

Compared with the prior art, the present invention has obvious advantages and beneficial effects. Specifically, it can be known from the above technical solutions.

1. The flow stopping valve is made of a membrane material. The inner and outer surfaces of the flow stopping valve are coated with a bactericidal substance. The rear-end outlet of the flow channel is a flat slit. When urine flows through the rear-end outlet, due to the extendibility of the membrane, the urine is drained when the urine opens up the rear-end outlet. When the urine is fully drained from the rear-end outlet, the rear-end outlet closes due to the extendibility of the membrane, thus preventing the backflow of the urine and preventing the patient from being infected. Compared with the conventional flow stopper, the invention has a simple structure, is convenient and practical, and can automatically prevent urine from flowing back.

2. The flow stopping valve adopts a double valve design, including the first flow stopping valve and the second flow stopping valve. The design of the double valve provides a double security to ensure that the urine will not flow back. Even if a small amount of urine flows back into the second flow stopping valve, the pressure of the urine is too small to open the first flow stopping valve, so to prevent the urine from flowing back.

In order to more clearly illustrate the structural features and effects of the present invention, embodiments of the present invention will now be described, by way of example only, with reference to the accompanying drawings.

### BRIEF DESCRIPTION OF THE DRAWINGS

FIG. 1 is a perspective view of a first embodiment of the present invention;

FIG. 2 is an exploded view of the first embodiment of the present invention;

FIG. 3 is a cross-sectional view of the first embodiment of the present invention;

FIG. 4 is a schematic view of a second embodiment of the present invention; and

FIG. 5 is an exploded view of the second embodiment of the present invention.

### DETAILED DESCRIPTION OF THE PREFERRED EMBODIMENTS

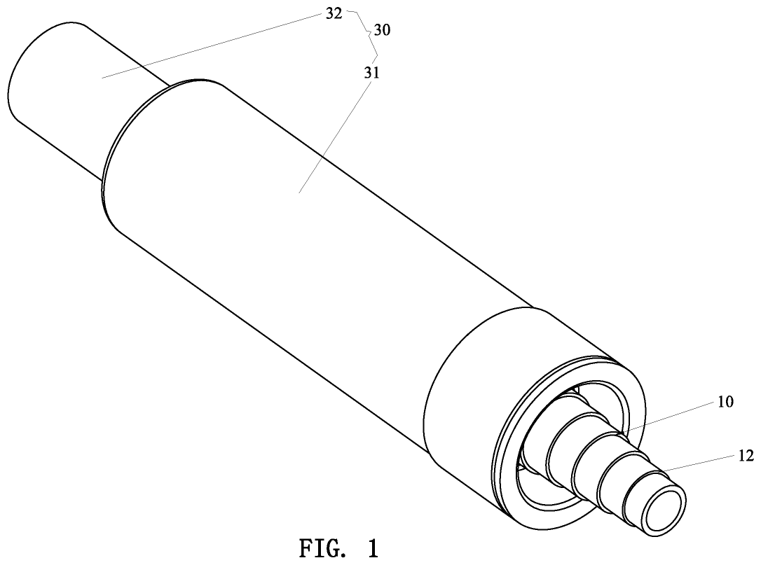

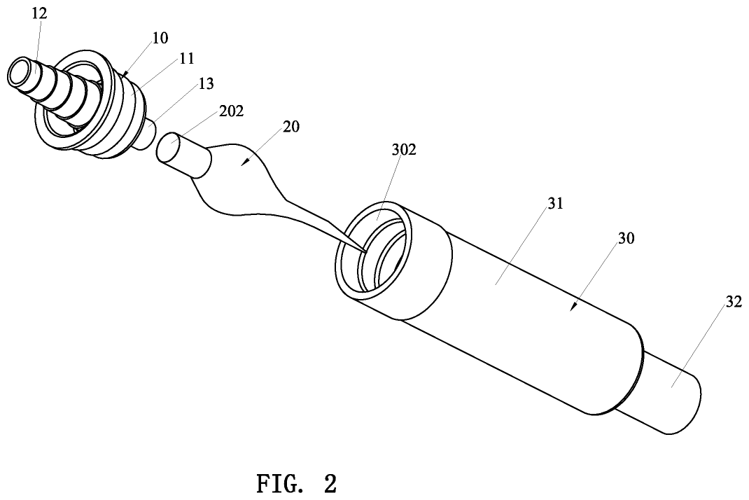

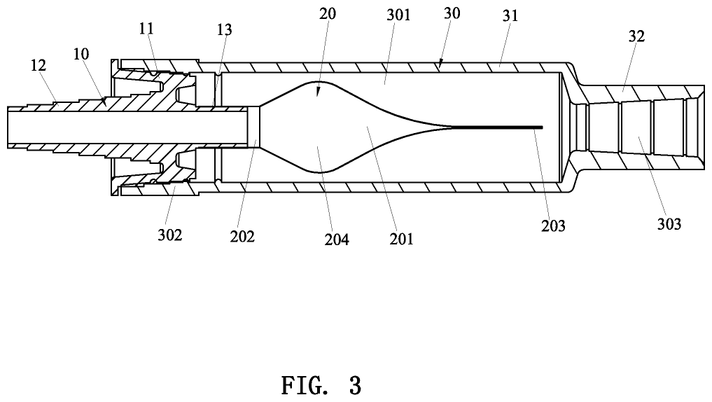

FIGS. 1 to 3 show the specific structure of a first embodiment of the present invention, comprising a urinary catheter connector 10, a flow stopping valve 20, and a urinary bag connector 30.

The urinary catheter connector 10 includes a base 11, a urinary catheter port 12 extending from the outer side of the base 11, and a valve port 13 extending from the inner side of the base 11. The urinary catheter port 12 is in communication with the valve port. A urinary catheter is connected to the urinary catheter port 12 for collecting the patient's urine.

The flow stopping valve 20 is made of a membrane material, such as TPE. The inner and outer surfaces of the flow stopping valve 20 are coated with a bactericidal substance. The bactericidal substance is used to kill the bacteria in the urine if the urine flows back to the urinary catheter, thereby reducing infection. The flow stopping valve 20 has a flow channel 201 passing through the front and rear ends of the flow stopping valve 20. The flow channel 201 is provided with a front-end inlet 202 and a rear-end outlet 203. The front-end inlet 202 is in communication with the valve port 13. Specifically, the valve port 13 has a cylindrical shape. Correspondingly, the front-end inlet 202 of the flow channel has a circular shape. The front-end inlet 202 is sleeved onto the valve port 13. The rear-end outlet 203 is a flat slit. In this embodiment, the flow stopping valve 20 is an integrally formed structure. When the patient urinates, the urine flows into the flow channel 201 from the front-end inlet 202. Due to the extendibility of the membrane, the rear-end outlet 203 is expanded by urine, and the urine flows out from the rear-end outlet 203. When the urine is fully drained from the rear-end outlet 203, the rear-end outlet 203 closes due to the extendibility of the membrane, thus preventing the backflow of the urine and preventing the patient from being infected.

The urinary bag connector 30 has a channel 301 passing through the front and rear ends of the urinary bag connector 30. The channel 301 is provided with a front-end port 302 and a rear-end port 303 connected to the tube of a urinary bag. The base 11 is hermetically connected to the front-end port 302. Specifically, the urinary catheter connector 10 is a pagoda-shaped connector. The outer surface of the base 11 has an external thread. Correspondingly, the inner wall of the front-end port 302 has an internal thread. The external thread is mated with the internal thread. The flow stopping valve 20 is located in the channel 301. Moreover, the rear-end outlet 203 of the flow channel 201 faces the rear-end port 303 of the channel 301. The urine bag connector 30 includes a body portion 31 and a tail portion 32 integrally extending from the rear end of the body portion 31. The front-end port 302 is located on the body portion 31. The rear-end port 303 is located on the tail portion 32. The inner wall of the tail portion 32 has an internal thread connected with the tube of the urine bag. The urine bag connector 30 and the urine catheter connector 10 are made of a bactericidal material.

The working principle of this embodiment is described in detail as follows:

When the patient urinates, the urine flows through the urinary catheter port 12, the valve port 13, the front-end inlet 202, the flow channel 201, the rear-end outlet 203 and the rear-end port 303 in sequence. When the urine flows through the rear-end outlet 203, due to the extendibility of the membrane, the urine is drained when the urine opens up the rear-end outlet 203. When the urine is fully drained from the rear-end outlet 203, the rear-end outlet 203 closes due to the extendibility of the membrane, thus preventing the backflow of the urine and preventing the patient from being infected.

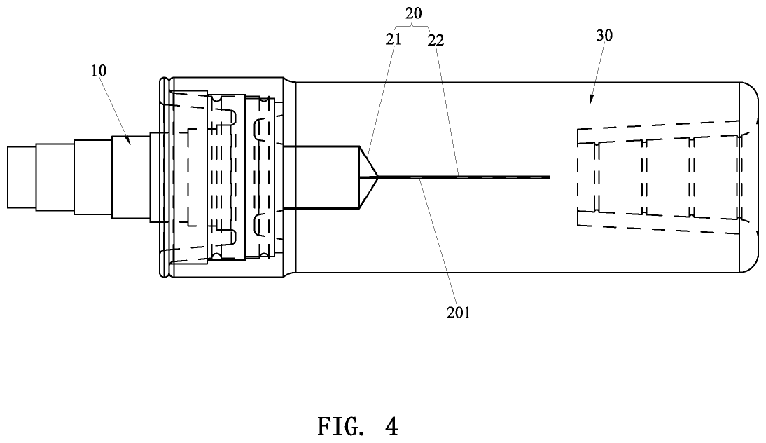

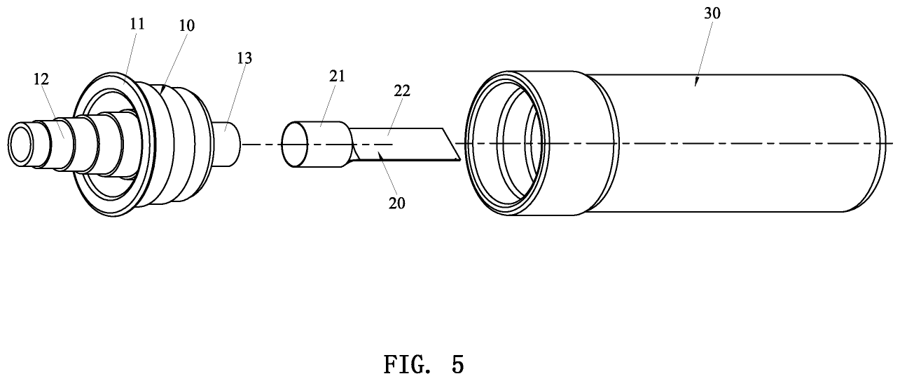

FIG. 4 and FIG. 5 show a second embodiment of the present invention. The second embodiment is substantially similar to the first embodiment with the exceptions described hereinafter.

In this embodiment, the flow stopping valve 20 includes a first flow stopping valve 21 and a second flow stopping valve 22 that are in communication with each other. The flow channel is formed inside the first flow stopping valve 21 and the second flow stopping valve 22. The front-end inlet of the first flow stopping valve 21 is in communication with the valve port 13. The rear-end outlet of the first flow stopping valve 21 is a flat slit. The junction of the interior of the front end and the interior of the rear end of the first flow stopping valve 21 is Y-shaped. The Y-shaped valve is automatically opened under the pressure of the urine for the urine to pass therethrough. The second flow stopping valve 22 has a flat shape. The second flow stopping valve 22 is sleeved onto the rear-end outlet of the first flow stopping valve 21. After the urine is drained, the first flow stopping valve 21 automatically closes, and the second flow stopping valve 22 also automatically closes. The design of the double valve provides a double security to ensure that the urine will not flow back. Even if a small amount of urine flows back into the second flow stopping valve 22, the pressure of the urine is too small to open the first flow stopping valve 21, so to prevent the urine from flowing back.

The features of the present invention are described below.

1. The flow stopping valve is made of a membrane material. The inner and outer surfaces of the flow stopping valve are coated with a bactericidal substance. The rear-end outlet of the flow channel is a flat slit. When the urine flows through the rear-end outlet, due to the extendibility of the membrane, the urine is drained when the urine opens up the rear-end outlet. When the urine is fully drained from the rear-end outlet, the rear-end outlet closes due to the extendibility of the membrane, thus preventing the backflow of the urine and preventing the patient from being infected. Compared with the conventional flow stopper, the invention has a simple structure, is convenient and practical, and can automatically prevent urine from flowing back.

2. The flow stopping valve adopts a double valve design, including the first flow stopping valve and the second flow stopping valve. The double valve design provides a double security to ensure that the urine will not flow back. Even if a small amount of urine flows back into the second flow stopping valve, the pressure of the urine is too small to open the first flow stopping valve, so to prevent the urine from flowing back.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A flow stopper, comprising a urinary catheter connector, a flow stopping valve, and a urinary bag connector;
the urinary catheter connector including a base, a urinary catheter port extending from an outer side of the base and a valve port extending from an inner side of the base, the urinary catheter port being in communication with the valve port;
the flow stopping valve being made of a membrane material, inner and outer surfaces of the flow stopping valve being coated with a bactericidal substance, the flow stopping valve having a flow channel passing through front and rear ends of the flow stopping valve, the flow channel being provided with a front-end inlet and a rear-end outlet, the front-end inlet being in communication with the valve port, the rear-end outlet being a flat slit;
the urinary bag connector having a channel passing through front and rear ends of the urinary bag connector, the channel being provided with a front-end port and a rear-end port connected to a tube of a urinary bag, the base being hermetically connected to the front-end port, the flow stopping valve being located in the channel, the rear-end outlet of the flow channel facing the rear-end port of the channel.

2. The flow stopper as claimed in claim 1, wherein the flow stopping valve is an integrally formed structure.

3. The flow stopper as claimed in claim 1, wherein the flow stopping valve includes a first flow stopping valve and a second flow stopping valve that are in communication with each other; the flow channel is formed inside the first flow stopping valve and the second flow stopping valve, a front-end inlet of the first flow stopping valve is in communication with the valve port, a rear-end outlet of the first flow stopping valve is a flat slit; the second flow stopping valve has a flat shape, and the second flow stopping valve is sleeved onto the rear-end outlet of the first flow stopping valve.

4. The flow stopper as claimed in claim 3, wherein a junction of an interior of a front end and an interior of a rear end of the first flow stopping valve is Y-shaped.

5. The flow stopper as claimed in claim 1, wherein the urinary catheter connector is a pagoda-shaped connector, an outer surface of the base has an external thread, an inner wall of the front-end port has an internal thread, and the external thread is mated with the internal thread.

6. The flow stopper as claimed in claim 1, wherein the valve port has a cylindrical shape, the front-end inlet of the flow channel has a circular shape, and the front-end inlet is sleeved onto the valve port.

7. The flow stopper as claimed in claim 1, wherein the urine catheter connector and the urine bag connector are made of a bactericidal material.

8. The flow stopper as claimed in claim 1, wherein the urine bag connector includes a body portion and a tail portion integrally extending from a rear end of the body portion, the front-end port is located on the body portion, the rear-end port is located on the tail portion, and an inner wall of the tail portion has an internal thread connected with the tube of the urine bag.
